(19) United States
(12) Patent Application Publication
(10) Pub. No.: US 2006/0009749 A1
(43) Pub. Date: Jan. 12, 2006

(54) Efficient diffuse light source assembly and method

(76) Inventors: Mark V. Weckwerth, Pleasanton, CA (US); Tobin C. Island, Oakland, CA (US); Robert E. Grove, Pleasanton, CA (US); Harvey I. Liu, Fremont, CA (US)

Correspondence Address: DLA PIPER RUDNICK GRAY CARY US, LLP, 2000 UNIVERSITY AVENUE, E. PALO ALTO, CA, 94303-2248, US

(21) Appl. No.: 11/157,275

(22) Filed: Jun. 20, 2005

Related U.S. Application Data
(63) Continuation-in-part of application No. 10/783,880, filed on Feb. 19, 2004.

(51) Int. Cl. A61B 18/20

(52) U.S. Cl. 606/9

(57) ABSTRACT

A diffuse light source assembly and method including a light source for generating forward propagating light, a solid lightguide disposed adjacent the light source, a diffuser and a back reflecting surface. The solid lightguide includes an input face for receiving the forward propagating light, a sidewall for conveying the forward propagating light via total internal reflection, and an output face for transmitting the forward propagating light. The diffuser is disposed adjacent the output face for diffusing the transmitted forward propagating light, wherein a portion of the forward propagating light is transformed into reverse propagating light, by at least one of the output face and the diffuser, that is conveyed by sidewall via total internal reflection and transmitted by the input face. The back reflecting surface is disposed adjacent the light source for reflecting the reverse propagating light back into the lightguide via the input face.

This application is a continuation-in-part of U.S. application Ser. No 10/783,880, filed Feb. 19, 2004, which is hereby incorporated herein by reference.

### FIELD OF THE INVENTION

The present invention relates to efficient, diffuse light sources, and more particularly to a laser diode assembly that efficiently produces a diffuse light output ideal for applications such as hair removal.

### BACKGROUND OF THE INVENTION

Currently, laser diodes are used to supply optical output for many varying types of applications. One such application is hair removal, although the present invention is not so limited. For many applications, one or more laser diodes are used in a single assembly in order to supply the requisite amount of optical power. However, the optical delivery systems used with the such optical assemblies can be inefficient in delivering the optical output to its intended target. This is especially true for applications that rely on optical diffusion. For example, in the application of hair removal, it is important to sufficiently diffuse the light before it exits the device to enhance safety, and to produce an even distribution of the light on the target. However, diffusing the light typically decreases substantially the optical output power, and can reduce the system efficiency below acceptable levels.

An ideal disk diffuser is one that converts an input beam, without loss, to an output beam having a Lambertian divergence distribution. A Lambertian divergence distribution is one where the intensity measured in the far field has a cos(Ø) dependence where Ø is the angle to the normal of the output face of the diffuser. Here, a source of light having a Lambertian divergence distribution is considered ideal since the “brightness” of such a beam becomes independent of Ø when the beam intensity has a cos(Ø) dependence. This is because the apparent size of the beam (or of any two dimensional, flat surface) varies perfectly with cos(Ø). Thus the decrease in apparent size with increasing viewing angle is exactly proportional to the decrease in intensity reaching the viewer.

There are several reasons why a non-ideal (real) diffuser is less than ideal. Most diffusers are either bulk diffusers or surface diffusers. Bulk diffusers are diffusers in which the scattering of the input beam occurs mainly within the volume of the diffusing material. An example of a bulk diffuser is PTFE (“Teflon”). Surface diffusers are diffusers that scatter, refract, reflect and/or diffract the light as it enters, exits, or reflects off of the diffuser. Examples of surface diffusers are etched glass, ground glass, and substrates patterned with diffraction features. Unless care is taken to provide an anti-reflection coating on the input face of the bulk or surface diffuser, there will generally be Fresnel reflections due to the change in index of refraction upon entering the diffuser that will reflect some of the light back towards the source. When the input beam is not perfectly collimated, it is difficult to provide an efficient anti-reflection coating since the performance of anti-reflection coatings is generally dependent on the incident angle. Fresnel reflections will also occur at the output face of the diffuser due to the change in refractive index as the light passes from the diffuser. Providing an effective anti-reflection coating at this interface is even more challenging since the light exiting the diffuser has been made even more diffuse (i.e., even less collimated). Thus, light will be reflected back towards the source from the exit face of the diffuser as well.

Bulk diffusers present an additional challenge in that the light may be scattered back towards the source within the scattering material itself. The amount of back-scattered light generally increases with the thickness of the bulk diffuser. Unfortunately, an output distribution that most closely matches a Lambertian distribution is achieved by increasing the thickness of the bulk diffuser to a point that a significant amount of light is scattered backwards toward the light source. Additionally, most diffusers are limited in size or there is an output aperture through which the output from the diffuser must pass. In these cases light scattered laterally within the diffusing material to the edge of the diffuser or light that is emitted from the diffuser outside of the emission aperture may also be lost. Another source for loss of light is absorption of the light within the diffuser. However, for many wavelengths this problem is not significant since diffuser materials can be found for many wavelengths that have negligible absorption.

Surface diffusers present a somewhat similar problem. A single surface diffuser often does not provide adequate scattering. Therefore, to achieve a greater level of scattering, multiple scattering surfaces must be used. Unfortunately, employing more scattering surfaces decreases the amount of transmitted light.

The efficiency of the diffuser is the fraction of the incident light that is transmitted by the diffuser. The designer of an optical system requiring a diffuse beam of light must often sacrifice the degree to which the output beam is Lambertian with the need for an efficient diffuser, or must employ the use of a more intense light source that may add size, expense and power consumption. This is especially true in optical systems that use a laser for the source of the light since laser outputs are generally fairly well collimated and must be diffused significantly in order to achieve a Lambertian divergence distribution. It is therefore imperative that the light reaching, and eventually transmitted beyond, the diffuser is maximized while maintaining the requisite degree to which the transmitted output is Lambertian.

### SUMMARY OF THE INVENTION

The present invention is a diffuse light source assembly that includes a light source for generating forward propagating light, a solid lightguide disposed adjacent the light source and having an input face for receiving the forward propagating light, a sidewall for conveying the forward propagating light via total internal reflection, and an output face for transmitting the forward propagating light, a diffuser disposed for diffusing the forward propagating light, and a back reflecting surface. A portion of the forward propagating light is transformed into reverse propagating light by the output face, which is conveyed by the sidewall via total internal reflection and transmitted by the input face. The back reflecting surface is disposed adjacent the light source for reflecting the reverse propagating light back into the lightguide via the input face.

In another aspect of the present invention, a diffuse light source assembly includes a light source for generating forward propagating light, a solid lightguide disposed adjacent the light source and having an input face for receiving the forward propagating light, a sidewall for conveying the forward propagating light via total internal reflection, and an output face for transmitting the forward propagating light, a diffuser disposed adjacent the output face for diffusing the transmitted forward propagating light, wherein a portion of the forward propagating light is transformed into reverse propagating light by the diffuser that is conveyed by the sidewall via total internal reflection and transmitted by the input face, and a back reflecting surface disposed adjacent the light source for reflecting the reverse propagating light back into the lightguide via the input face.

In yet another aspect of the present invention, a method of generating diffuse light that includes generating forward propagating light, conveying the forward propagating light using a solid lightguide having an input face for receiving the forward propagating light, a sidewall for conveying the forward propagating light via total internal reflection, and an output face for transmitting the forward propagating light, diffusing the forward propagating light using a diffuser wherein a portion of the forward propagating light is transformed into reverse propagating light by the output face which is conveyed by the sidewall via total internal reflection and transmitted by the input face, and reflecting the reverse propagating light back into the lightguide through the input face using a back reflecting surface disposed adjacent the light source.

In yet one more aspect of the present invention, a method of generating diffuse light includes generating forward propagating light, conveying the forward propagating light using a solid lightguide having an input face for receiving the forward propagating light, a sidewall for conveying the forward propagating light via total internal reflection, and an output face for transmitting the forward propagating light, diffusing the forward propagating light using a diffuser, wherein a portion of the forward propagating light is transformed into reverse propagating light by the diffuser which is conveyed by the sidewall via total internal reflection and transmitted by the input face, and reflecting the reverse propagating light back into the lightguide through the input face using a back reflecting surface disposed adjacent the light source.

Other objects and features of the present invention will become apparent by a review of the specification, claims and appended figures.

### BRIEF DESCRIPTION OF THE DRAWINGS

FIG. 1A is an exploded perspective view of the diffuse light source assembly of the present invention.

FIG. 1B is an exploded side view of the diffuse light source assembly of the present invention.

FIG. 2 is a cross-sectional side view of the diffuse light source assembly of the present invention.

FIG. 3 is a cross-sectional side view of the optical cavity of the present invention, illustrating forward propagating light, reverse propagating light, and light reflected back toward the forward propagating direction.

FIG. 4 is a top view of the mask member of the present invention.

FIG. 5 is a system schematic view illustrating an optical fiber delivery system used with the present invention.

FIGS. 6A and 6B are side views illustrating embodiments with reflective-type diffusers adjacent the laser diodes or optical fiber delivery system, respectively.

FIG. 7 is a side cross-sectional view illustrating diffusers that are integrally formed as part of the input and output faces of the solid lightguide.

### DETAILED DESCRIPTION OF THE PREFERRED EMBODIMENTS

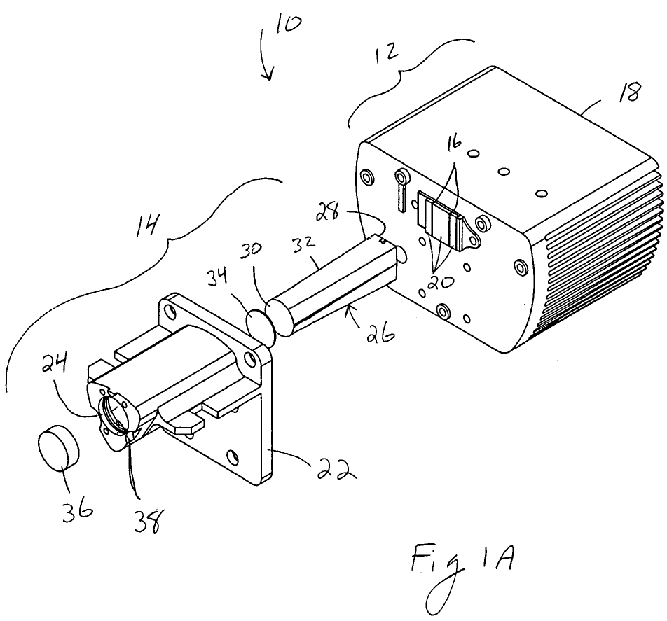

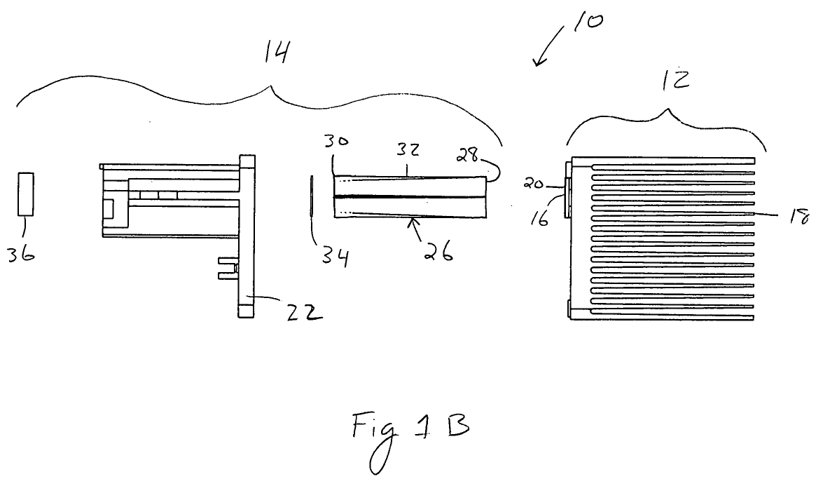

The present invention is a diffuse light source assembly and method that efficiently generates and delivers sufficiently diffuse optical output to intended targets. The diffuse light source assembly 10 of the present invention is illustrated in FIGS. 1 and 2, and includes a laser diode assembly 12 and a delivery assembly 14.

Laser diode assembly 12 includes one or more laser diodes 16 mounted onto a heat sink 18. In the embodiment illustrated in the figures, a pair of laser diode bars 16 mounted between three mounting blocks 20 are used to create an optical output. The mounting blocks 20 are used to position the laser diode bars 16 during manufacture, and are made of electrically conductive metal to complete the electrical circuit that operates the laser diodes 16. Various techniques of mounting of laser diodes, such as laser diodes separated by mounting blocks or laser diodes placed in grooves formed in a monolithic substrate, are well known in the art, and are not further described herein. However, according to the present invention, the top surfaces of mounting blocks 20 are made of, or plated with, a highly reflective material such as gold, as explained further below.

The delivery assembly 14 includes a barrel shaped housing 22 with an elongated cavity 24 therein. A lightguide 26 (sometimes termed a “mixer”) made of an elongated block of transparent material (e.g. boro-silica glass, acrylic, sapphire, etc.) is disposed in the elongated cavity 24. In this embodiment, lightguide 26 includes a rectangular shaped input face 28 positioned adjacent the laser diodes 16 and a circular shaped output face 30, with a sidewall 32 extending therebetween having a cross-sectional shape that gradually changes from rectangular to circular. The gradual change is such that light traveling down the lightguide 26 will be reflected with little or no loss by total internal reflection (TIR), as explained further below. A diffuser 34 is disposed over the output face 30, and is preferably made of PTFE, opal glass, or similar diffusive material. In a preferred embodiment, diffuser 34 is displaced from output face 30 by a small air gap. A protective transparent window 36 is disposed over the diffuser 34, and is preferably made of sapphire. Preferably, the cavity 24 includes a stepped shoulder 38 in which the window 36 is held by adhesive and/or friction fit. The housing 22 is preferably bolted onto the heat sink 18 to secure the delivery assembly 14 to the laser diode assembly 12. Optionally the barrel may be maintained at a different temperature than the heatsink, by, for example, placing a thermoelectric module 46 between the housing 22 and the heatsink 18 as shown in FIG. 2.

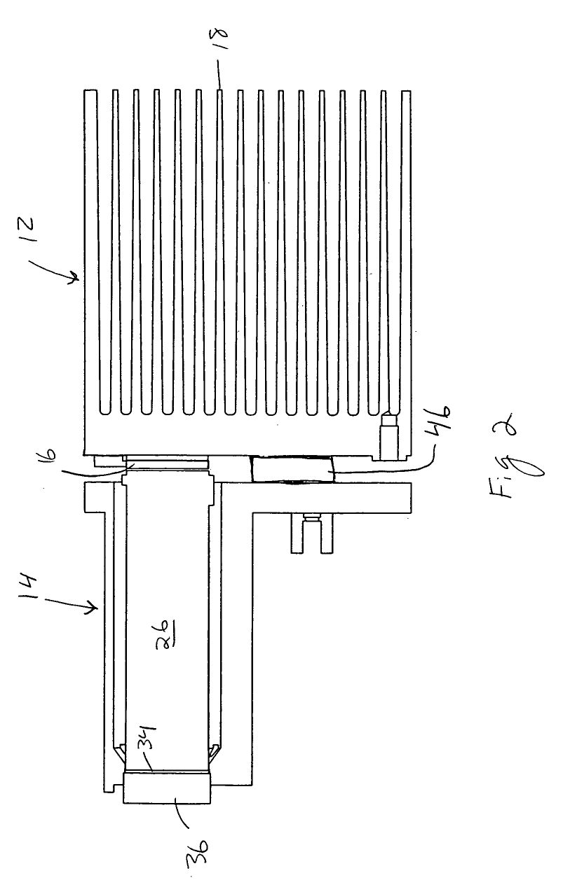

The operation of the diffuse light source assembly 10 is illustrated in FIG. 3, where light output 40 emitted by the laser diodes 16 enters the lightguide 26 through input face 28. The lightguide 26 conveys the light output 40 to the output face 30 via TIR from sidewall 32. The light output 40 exits the lightguide through output face 30, where it is subjected to diffusion by diffuser 34. The diffused light output 40 then exits the delivery assembly 14 after passing through window 36.

There are several sources of light loss in the optical configuration of FIG. 3. Specifically, some of the forward propagating light (i.e. propagating away from the laser diodes 16) is reflected back toward the laser diodes (i.e. in a reverse propagating direction) by the input face 28, by the output face 30, by the surfaces and within diffuser 34, and by the surfaces of window 36. Much of this reverse propagating light is conveyed by the lightguide 26 (via TIR) back to the laser diode assembly 12. Therefore, to re-use the reverse propagating light 42 according the present invention, that portion of the laser diode assembly 12 receiving this reverse propagating light is formed of or coated with a highly reflective material, essentially creating a back reflective surface 44 around the laser diodes 16. The term “reflective” is used herein to refer not only to specular reflection but also diffuse reflection or remission of light. Thus, an optical cavity is formed by back reflective surface 44, lightguide 26, diffuser 34 and window 36. For the embodiment described above, this means that mounting blocks 20 are made of or coated with a highly reflective material, to reflect the reverse propagating light 42 back in the forward propagating direction, whereby much of this light will be diffused by diffuser 34 and emitted by window 36, thus increasing the efficiency of the system. This process of reflecting reverse propagating light back towards the diffuser is repeated until all of the light is transmitted or lost to parasitic absorption within the lightguide 26, diffuser 34 or back reflective surface 44. There may also be a slight loss of light due to lateral scattering within the diffuser where light may be lost to the edge of the diffuser 34 or scattered backwards outside of the lightguide 26; or lost due to the gap between the input face 28 of the lightguide 26 and the back reflective surface 44. (Some gap may be necessary to reduce the intensity of the laser diode light on the input face 28 of the lightguide 26, although in general this gap should be minimized to reduce lateral loss of light.) Another source for loss of light may be re-absorption by the laser diodes 16 when reverse propagating light is incident directly on the laser diodes.

With the present invention, a nearly Lambertian output beam is realized with very little loss of light. To optimize the design of this highly efficient Lambertian diffuser, the thickness of the diffuser (or in the case of a surface diffuser, the number of surfaces) can be reduced so as to minimize the amount of light that is scattered laterally. The diffuser thickness (or number of surfaces) that is required for adequate scattering may be less than what would be required for a single pass of light since light that has be redirected back to the diffuser by reflections off the mounting blocks in subsequent passes will likely be more diffuse than the initial beam of light.

It is also important for an optimal design to minimize the amount of absorption within the diffuser material and the absorption at the lightguide sidewall 32. Since the light returned from the diffuser into the cavity may be nearly Lambertian (and therefore very divergent), the reflected light will impinge upon the sidewall 32 many times if the length of the lightguide 26 is large. Multiple reflections from imperfectly reflecting cavity walls will absorb some of the back-scattered light. This is why a solid lightguide 26 using TIR to reflect the light along the lightguide is ideal and preferred over a hollow lightguide relying on surface reflections. So long as the angle of incidence is high enough (given the refractive index of material), losses are minimized or even essentially eliminated, even though the diffuser 34 creates high angle reverse propagating light. In order to collect and return as much of the light as possible, any gaps between the lightguide 26, diffuser 34 and back reflective surface 44 should be minimized. Further, it is desirable to minimize the size of the laser diodes 16 relative to the back reflective surface 44 so that the maximum amount of light is reflected and the minimum amount of light is absorbed by the laser diodes. Further, the reflective surface 44 should extend over an area at least as large (and preferably somewhat larger) than the area of the input face 28 of the lightguide 26. It is also important for the lightguide 26 to have sufficient length to spatially mix the light from the laser diodes, a length of several centimeters being typically sufficient.

An embodiment of the present invention has been reduced to practice, using a gold reflective coating on the mounting blocks 20 to form back reflecting surface 44 (which is 94% reflective at 800 nm), a 0.015″ thick PTFE disk for the diffuser 34, an acrylic solid lightguide 26, and a pair of laser diode bars having a total area of about 2×1 cm×0.03 cm. The output window 36 is about 1 cm in diameter. The sidewall 32 is not perfectly orthogonal to the back reflective surface or diffuser disk. However, no portions of the sidewall 32 exceed about 7 degrees away from a perfect orthogonal orientation relative to the back reflective surface or diffuser disk, so that light will not leak out sidewall 32.

The shape of lightguide is such that a generally rectangular distribution of the light output 40 is transformed to a generally circular distribution. It should be noted, however, that the lightguide 26 need not have a rectangular input face 28 and a circular output face 30. Such a configuration is preferred, however, because a round optical output cross-section can be achieved at the window 36 (permitting use of a conventional round output window 36) while using a back reflective surface 44 that is not any longer than the laser diodes 16. That is, the reflective mounting blocks 20 can have the same length as the laser diodes 16 (a desirable feature for manufacturability) and yet completely fill the input face 28 for reflecting all of the reverse propagating light. Alternatively, a back reflective surface of greater dimension than input face 28, or a mask as described below, can be used so that lightguide 26 can have a uniform cross sectional shape. In addition and/or alternately, the lightguide sidewall 32 can be tapered, so that input face 28 can have a different desired cross-sectional area compared to output face 30. The higher the refractive index of material used to form lightguide 26, the greater the amount of taper that can implemented before significant amounts of light leakage out of lightguide 26 occur (due to light rays striking the sidewall 32 below the critical angle for TIR).

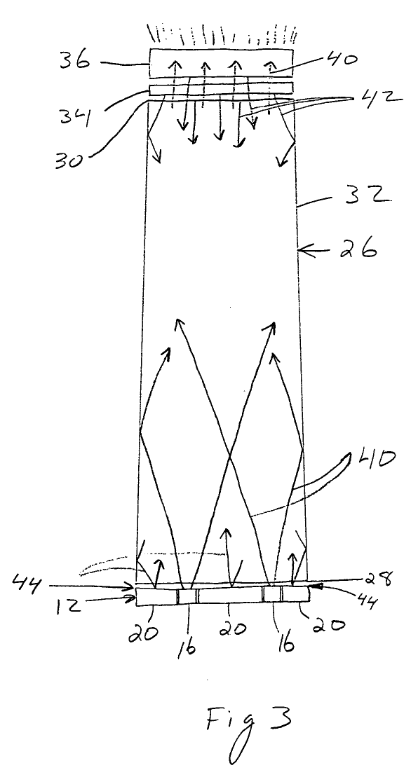

FIG. 4 illustrates an alternative embodiment of the present invention, which includes a mask 50 placed over the laser diodes 16 and mounting blocks 20, and having an upper surface that serves as the back reflective surface 44. The mask includes apertures 52 through which the light output 40 from the laser diodes 16 passes. To maximize the efficiency of the diffuser assembly, apertures 52 are preferably as narrow as possible without blocking significant light from the laser diodes, which will require careful alignment of the mask apertures with the laser diodes. The mask can be sandwiched between assemblies 10 and 12, attached to the lightguide input face 28, and/or attached to the laser diode mounting blocks 20. It should be noted that the use of reflective mounting blocks 20, rather than mask 50, eliminates this alignment task and thus is a key advantage of using reflective mounting blocks.

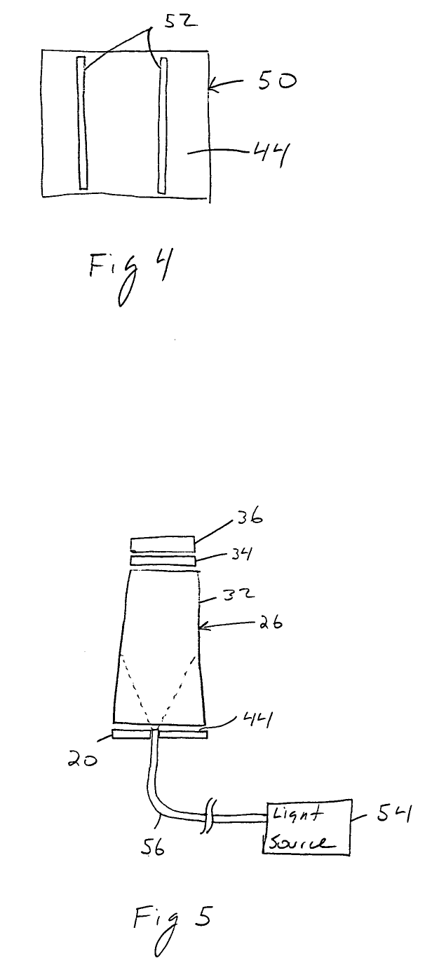

It should be noted that other light sources can be used instead of one or more laser diodes. For example, other solid state lasers (e.g. Nd:YAG, etc.), gas lasers (e.g. argon, krypton, etc.) or dye laser lasers, or even a flash lamp, can be used to generate light output 40. Because these types of light sources tend to be less compact than laser diodes, any light source 54 used as part of the present invention (including laser diodes, solid state lasers, gas lasers, flash lamps, etc.) can include a delivery system such as an optical fiber 56 as shown in FIG. 5. In that case, the back reflective surface 44 would be disposed at the output of the delivery system.

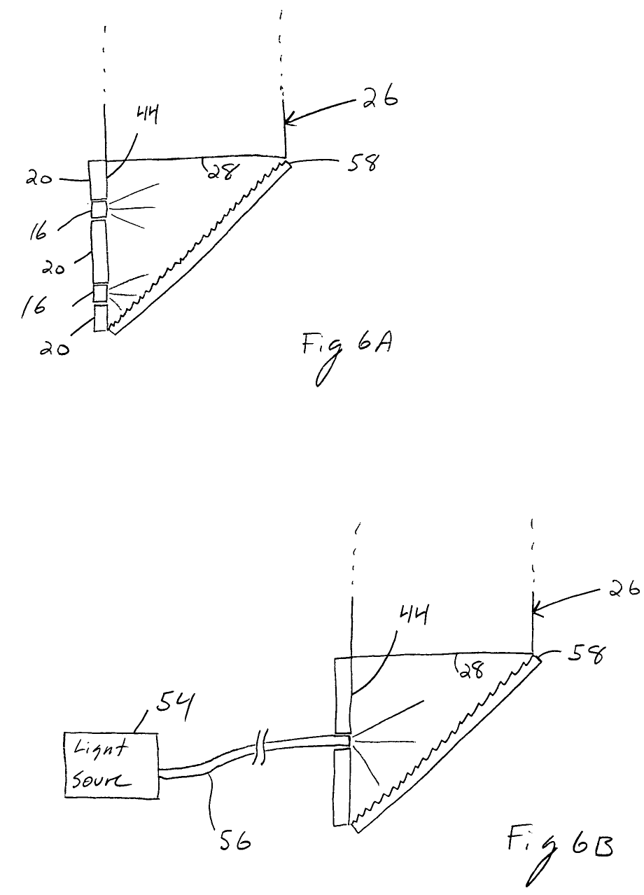

FIGS. 6A and 6B illustrate another alternative embodiment of the present invention, which utilizes a reflective-type diffuser 58 instead of a transmissive-type diffuser. In this embodiment, the output of the laser diodes 16 (as shown in FIG. 6A), or an optical fiber (as shown in FIG. 6B), is directed to a reflective-type diffuser 58, which either has an irregular reflecting surface or includes a diffusive material through which the light passes before and/or after reflection, that both reflects and diffuses the light, and directs the diffused light output to the lightguide 26. Any light directed back toward the laser diodes 16 or optical fiber 56 will be reflected by back reflective surface 44 disposed adjacent the laser diode output facets or the optical fiber's delivery end. Reflective diffusers can be made of a highly scattering material such as PTFE, or a commercially available material termed Spectralon (LabSphere, Inc., North Sutton, N.H.); or a scattering material applied to a reflecting surface, such as Duraflect (also available from LabSphere, Inc.).

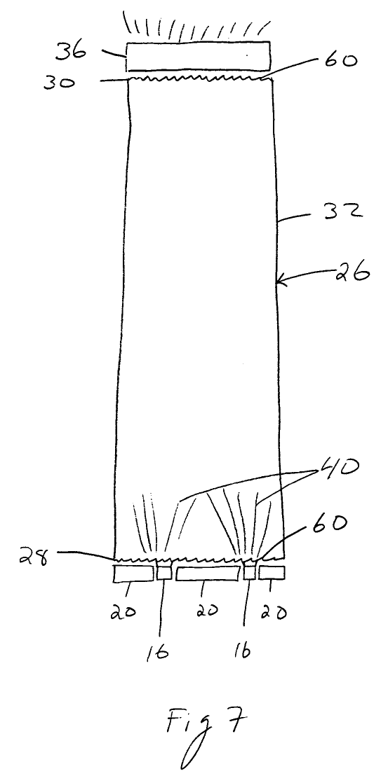

FIG. 7 illustrates yet another alternative embodiment, where the lightguide input and/or output faces 28/30 integrally include diffusers, by including a diffusive material on these faces. If the input and/or output faces 28/30 produce a sufficient amount of diffusion for the light, separate diffuser 34 may be eliminated.

It should be noted that, as used herein, the terms “over” and “on” both inclusively include “directly on” (no intermediate materials, elements or space disposed therebetween) and “indirectly on” (intermediate materials, elements or space disposed therebetween). Likewise, the term “adjacent” includes “directly adjacent” (no intermediate materials, elements or space disposed therebetween) and “indirectly adjacent” (intermediate materials, elements or space disposed therebetween).

It is to be understood that the present invention is not limited to the embodiment(s) described above and illustrated herein, but encompasses any and all variations falling within the scope of the appended claims. For example, materials, and numerical examples described above are exemplary only, and should not be deemed to limit the claims. The back reflective surface is adjacent the laser diodes, meaning that the output facets of the laser diodes 16 can be flush with, be recessed relative to, or extend slightly beyond, the back reflective surface 44 (i.e. laser diodes can be even with, disposed outside of, or extend into, the optical cavity formed by back reflective surface 44, lightguide 26, diffuser 34 and window 36). Back reflective surface 44 can be a spectral reflective surface (e.g., polished or polished and plated) or simply coated without polishing, creating a diffuse reflective surface that efficiently remits light.

What is claimed is:

1. A diffuse light source assembly, comprising:
a light source for generating forward propagating light;
a solid lightguide disposed adjacent the light source and having an input face for receiving the forward propagating light, a sidewall for conveying the forward propagating light via total internal reflection, and an output face for transmitting the forward propagating light;
a diffuser disposed for diffusing the forward propagating light;
wherein a portion of the forward propagating light is transformed into reverse propagating light by the output face, which is conveyed by the sidewall via total internal reflection and transmitted by the input face; and
a back reflecting surface disposed adjacent the light source for reflecting the reverse propagating light back into the lightguide via the input face.

2. The diffuse light source assembly of claim 1, further comprising:
a transparent window disposed for receiving the diffused forward propagating light.

3. The diffuse light source assembly of claim 1, further comprising:
one or more mounting blocks disposed adjacent the light source, wherein the mounting blocks have upper surfaces formed or coated with a reflective material to form the back reflecting surface.

4. The diffuse light source assembly of claim 3, wherein the reflective material is gold.

5. The diffuse light source assembly of claim 3, wherein the mounting blocks are in electrical contact with the light source such that electrical current flowing through the mounting blocks flows through the light source to generate the forward propagating light.

6. The diffuse light source assembly of claim 1, wherein the lightguide sidewall is tapered such that the input face has a different area than that of the output face.

7. The diffuse light source assembly of claim 1, wherein the lightguide has a rectangular cross section adjacent the input face that gradually changes to a round cross section adjacent the output face.

8. The diffuse light source assembly of claim 1, wherein the light source comprises one or more laser diodes.

9. The diffuse light source assembly of claim 1, further comprising:
a heat sink on which the light source is mounted; and
one or more mounting blocks disposed adjacent the light source and mounted on the heat sink, wherein the mounting blocks have upper surfaces formed or coated with a reflective material to form the back reflecting surface.

10. The diffuse light source assembly of claim 1, further comprising:
a heat sink on which the light source is mounted; and
a housing having a cavity in which the diffuser and lightguide are disposed, wherein the housing is mounted to the heat sink.

11. The diffuse light source assembly of claim 10, wherein the housing includes a transparent window at least partially mounted in the cavity by friction fit for receiving the diffused forward propagating light.

12. The diffuse light source assembly of claim 10, further comprising:
a thermoelectric cooler for transferring heat from the housing and to the heat sink.

13. The diffuse light source assembly of claim 1, further comprising:
a mask member disposed adjacent the lightguide input face, wherein the mask member includes:
at least one aperture through which the forward propagating light from the light source passes, and
a top surface facing the input face that comprises the back reflecting surface.

14. The diffuse light source assembly of claim 1, wherein the light source includes an optical fiber having an output end disposed adjacent the lightguide input face, and wherein the back reflecting surface is disposed adjacent the optical fiber output end.

15. The diffuse light source assembly of claim 1, wherein the diffuser is disposed adjacent the output face for diffusing the forward propagating light transmitted by the output face.

16. The diffuse light source assembly of claim 1, wherein the diffuser is disposed adjacent the input face for diffusing the forward propagating light from the light source and directing the diffused forward propagating light into the input face.

17. The diffuse light source assembly of claim 1, wherein the diffuser is integrally formed as part of at least one of the input face and the output face.

18. A diffuse light source assembly, comprising:
a light source for generating forward propagating light;
a solid lightguide disposed adjacent the light source and having an input face for receiving the forward propagating light, a sidewall for conveying the forward propagating light via total internal reflection, and an output face for transmitting the forward propagating light;
a diffuser disposed adjacent the output face for diffusing the transmitted forward propagating light;
wherein a portion of the forward propagating light is transformed into reverse propagating light by the diffuser that is conveyed by the sidewall via total internal reflection and transmitted by the input face; and
a back reflecting surface disposed adjacent the light source for reflecting the reverse propagating light back into the lightguide via the input face.

19. The diffuse light source assembly of claim 18, further comprising:
a transparent window disposed adjacent the diffuser for receiving the diffused forward propagating light.

20. The diffuse light source assembly of claim 19, further comprising:
one or more mounting blocks disposed adjacent the light source, wherein the mounting blocks have upper surfaces formed or coated with a reflective material to form the back reflecting surface.

21. The diffuse light source assembly of claim 20, wherein the reflective material is gold.

22. The diffuse light source assembly of claim 20, wherein the mounting blocks are in electrical contact with the light source such that electrical current flowing through the mounting blocks flows through the light source to generate the forward propagating light.

23. The diffuse light source assembly of claim 18, wherein the lightguide sidewall is tapered such that the input face has a different area than that of the output face.

24. The diffuse light source assembly of claim 18, wherein the lightguide has a rectangular cross section adjacent the input face that gradually changes to a round cross section adjacent the output face.

25. The diffuse light source assembly of claim 18, wherein the light source comprises one or more laser diodes.

26. The diffuse light source assembly of claim 18, further comprising:
a heat sink on which the light source is mounted; and
one or more mounting blocks disposed adjacent the light source and mounted on the heat sink, wherein the mounting blocks have upper surfaces formed or coated with a reflective material to form the back reflecting surface.

27. The diffuse light source assembly of claim 18, further comprising:
a heat sink on which the light source is mounted; and
a housing having a cavity in which the diffuser and lightguide are disposed, wherein the housing is mounted to the heat sink.

28. The diffuse light source assembly of claim 27, wherein the housing includes a transparent window at least partially mounted in the cavity by friction fit for receiving the diffused forward propagating light.

29. The diffuse light source assembly of claim 27, further comprising:
a thermoelectric cooler for transferring heat from the housing and to the heat sink.

30. The diffuse light source assembly of claim 18, further comprising:
a mask member disposed adjacent the lightguide input face, wherein the mask member includes:
at least one aperture through which the forward propagating light from the light source passes, and
a top surface facing the input face that comprises the back reflecting surface.

31. The diffuse light source assembly of claim 18, wherein the light source includes an optical fiber having an output end disposed adjacent the lightguide input face, and wherein the back reflecting surface is disposed adjacent the optical fiber output end.

32. A method of generating diffuse light, comprising:
generating forward propagating light;
conveying the forward propagating light using a solid lightguide having an input face for receiving the forward propagating light, a sidewall for conveying the forward propagating light via total internal reflection, and an output face for transmitting the forward propagating light;
diffusing the forward propagating light using a diffuser;
wherein a portion of the forward propagating light is transformed into reverse propagating light by the output face, which is conveyed by the sidewall via total internal reflection and transmitted by the input face; and
reflecting the reverse propagating light back into the lightguide through the input face using a back reflecting surface disposed adjacent the light source.

33. The method of claim 32, further comprising:
transmitting the diffused forward propagating light through a transparent window.

34. The method of claim 32, wherein one or more mounting blocks are disposed adjacent to and in electrical contact with the light source, the method further comprising:
applying a voltage to the mounting blocks such that electrical current flows through the mounting blocks and through the light source to generate the forward propagating light.

35. The method of claim 34, wherein the reflecting of the reverse propagating light is performed using reflective material formed or coated on upper surfaces of the mounting blocks.

36. The method of claim 32, wherein the reflecting of the reverse propagating light is performed using a mask member disposed adjacent the input face, wherein the mask member comprises:
at least one aperture through which the forward propagating light passes, and a top surface facing the input face that comprises the back reflecting surface.

37. The method of claim 32, wherein the diffuser is disposed adjacent the output face for the diffusing of forward propagating light transmitted by the output face.

38. The method of claim 32, wherein the diffuser is disposed adjacent the input face for the diffusing of the forward propagating light and for directing the diffused forward propagating light into the input face.

39. The method of claim 32, wherein the diffuser is integrally formed as part of at least one of the input face and the output face.

40. A method of generating diffuse light, comprising:
generating forward propagating light;
conveying the forward propagating light using a solid lightguide having an input face for receiving the forward propagating light, a sidewall for conveying the forward propagating light via total internal reflection, and an output face for transmitting the forward propagating light;
diffusing the forward propagating light using a diffuser;
wherein a portion of the forward propagating light is transformed into reverse propagating light by the diffuser which is conveyed by the sidewall via total internal reflection and transmitted by the input face; and
reflecting the reverse propagating light back into the lightguide through the input face using a back reflecting surface disposed adjacent the light source.

41. The method of claim 40, further comprising:
transmitting the diffused forward propagating light through a transparent window.

42. The method of claim 40, wherein one or more mounting blocks are disposed adjacent to and in electrical contact with the light source, the method further comprising:
applying a voltage to the mounting blocks such that electrical current flows through the mounting blocks and through the light source to generate the forward propagating light.

43. The method of claim 42, wherein the reflecting of the reverse propagating light is performed using reflective material formed or coated on upper surfaces of the mounting blocks.

44. The method of claim 40, wherein the reflecting of the reverse propagating light is performed using a mask member disposed adjacent the input face, wherein the mask member comprises:
at least one aperture through which the forward propagating light passes, and a top surface facing the input face that comprises the back reflecting surface.
